(19) United States
(12) Patent Application Publication
(10) Pub. No.: US 2009/0136558 A1
(43) Pub. Date: May 28, 2009

(54) Anti-Restenosis Coatings and Uses Thereof

(75) Inventors: Tong Li Williams, Shrewsbury, MA (US); Tim Wu, Shrewsbury, MA (US)

Correspondence Address: CHOATE, HALL & STEWART LLP, TWO INTERNATIONAL PLACE, BOSTON, MA, 02110, US

(73) Assignee: VASOTECH, INC., Lowell, MA (US)

(21) Appl. No.: 12/209,104

(22) Filed: Sep. 11, 2008

Related U.S. Application Data
(63) Continuation-in-part of application No. 11/144,917, filed on Jun. 6, 2005, now abandoned.
Application No. 11/144,917 is a continuation-in-part of application No. 11/843,528, filed on Aug. 22, 2007.
(60) Provisional application No. 60/578,219, filed on Jun. 8, 2004.
(60) Provisional application No. 60/823,168, filed on Aug. 22, 2006.

(51) Int. Cl. A61F 2/00; A61K 31/4355; A61K 31/337

(52) U.S. Cl. 424/423; 514/291; 514/449

(57) ABSTRACT

The present invention provides coatings or coating compositions for implantable or insertable medical devices containing one or more polymers and a combination of an immunosuppressant agent and an anti-neoplastic agent. In some embodiments, the coatings or coating compositions of the invention control sustained-release of the immunosuppressant agent and the anti-neoplastic agent for at least about 4 weeks. The present invention also provides implantable or insertable medical devices and other drug delivery or eluting systems containing a coating or coating composition of the invention and uses thereof.

### CROSS-REFERENCE TO RELATED APPLICATIONS

This application is a continuation-in-part of the U.S. patent application Ser. No. 11/144,917, filed on Jun. 6, 2005, which claims the benefit of U.S. provisional application No. 60/578,219, filed on Jun. 8, 2004, the disclosures of all of which are hereby incorporated by reference in their entireties. This application is also a continuation-in-part of the U.S. patent application Ser. No. 11/843,528, filed on Aug. 22, 2007, which claims the benefit of U.S. provisional application No. 60/823,168, filed on Aug. 22, 2006, the disclosures of all of which are hereby incorporated by reference in their entireties.

### BACKGROUND OF THE INVENTION

Coronary Artery Disease (CAD) is the chronic coronary artery blocking/narrowing caused by neointima hyperplasia inside the arterial wall. It has been the number one killer in the Unite State since 1900 and still remains the most common cause of death in the western world despite the therapeutic advances. Approximately 14 million Americans have CAD, and 500,000 people die from acute myocardial infarction and one million more survive but with a 1.5 to 15 times greater risk of mortality or morbidity than the rest of the population each year. The annual medicare cost for the disease is in excess of $112 billion. The current level of certain predictors of heart disease risk, such as obesity, diabetes, and smoking, suggest that this will continue to be a significant public health issue for the foreseeable future.

Coronary artery bypass surgery, as a curative approach to coronary heart disease has been proven effective; however, high mortality, morbidity and economic cost have promoted a steady development of less invasive therapies. Two such therapies, Percutaneous Transluminal Coronary Artery Angioplasty (PTCA) and Coronary Artery Stenting (CAS) have experienced dramatic growth over the past 25 years.

PTCA involves insertion of an expandable balloon catheter against a primary atherosclerostic plaque or secondary restenotic lesion to increase vessel patency and blood flow. Clinical stenting was introduced in 1986 with the Wallstent to repair abrupt closure after PTCA, and has revolutionized interventional cardiology. In CAS, the stent (a tiny metal scaffolding) functions to brace the vessel wall and reduce the risk of restenosis following angioplasty. Common indications for PTCA and/or stenting are angina or acute myocardial infarction in vessel diameter of >3 mm.

According to the American Heart Association, 1.3 million patients underwent PTCA procedure in 1997 and half required stent placement. This trend, stenting coupled with PTCA, is growing at a rate of 20% annually, especially with recent development of drug delivery stent. The total direct costs for these life saving procedures is over $2 billion annually.

Restenosis, the re-narrowing of opened artery after stenting or PTCA procedure, is due to a proliferative response of the intima, a layer of cells that line the lumen of the vessel, composed of connective tissue and smooth muscle cells (SMC). In restenosis, vascular neointimal hyperplasia results in complete blockage of the original artery and insufficient oxygenation of cardiac tissue, leading to cardiac arrhythmia or cardiac arrest. Restenosis has been the biggest problem in Percutaneous Coronary Intervention (PCI) until the recently successful development of drug coated stents. Initially, the restenosis rate in PTCA procedure is as high as over 50% within six month post balloon dilation. Stenting lowered this number to 20-30%. However, restenosis in patients with high risk such as small vessels, diabetes, and long diffusion diseased arteries still remains unacceptablely high (30%-60% in bare metal stents and 6%-18% in drug coated stents).

Therefore, there remains a great need for improved anti-restenosis drugs and drug delivery systems.

### SUMMARY OF THE INVENTION

The present invention provides improved drugs and drug delivery systems for the effective prevention and/or treatment of restenosis and other diseases, disorders and conditions associated with hyperproliferation.

The present invention encompasses the discovery that a composition containing certain polymers and a combination of an anti-neoplastic agent and an immunosuppressant agent can be used as a coating for implantable medical devices that effectively controls sustained release of the anti-neoplastic agent and the immunosuppressant agent. The present invention also encompasses the finding that medical devices coated with such a coating are surprisingly effective in inhibiting, preventing, and/or delaying the onset of hyperproliferative conditions such as restenosis in vivo. The present invention therefore provides, among other things, coatings or coating compositions for medical devices comprising an immunosuppressant agent, an anti-neoplastic agent, and one or more polymers. The present invention further provides medical devices coated with inventive coatings according to the invention and other drug delivery or eluting systems and methods of their uses.

In one aspect, the present invention provides coatings or costing compositions for implantable or insertable medical devices comprising an immunosuppressant agent, an anti-neoplastic agent and one or more polymers, wherein the coatings are characterized with sustained-release of the immunosuppressant agent and anti-neoplastic agent for at least about 4 weeks (e.g., at least 5 weeks, 6 weeks, 7 weeks, 8 weeks, 9 weeks, 10 weeks, 11 weeks, 12 weeks, or longer).

In some embodiments, suitable immunosuppressant agent is sirolimus or a prodrug or analog thereof. In some embodiments, suitable immunosuppressant agents are selected from zotarolimus, tacrolimus, everolimus, biolimus, pimecrolimus, supralimus, temsirolimus, TAFA 93, invamycin, neuroimmunophilins, or combinations or analogs thereof. In some embodiments, suitable anti-neoplastic agent is paclitaxel or a prodrug or analog thereof. In some embodiments, suitable anti-neoplastic agent is selected from carboplatin, vinorelbine, doxorubicin, gemcitabine, actinomycin-D, cisplatin, camptothecin, 5-fluorouracil, cyclophosphamide, 1-β-D-arabinofuranosylcytosine, or combinations or analogs thereof. In some embodiments, the anti-neoplastic agent and immunosuppressant agent are present in a ratio, by weight, ranging from about 1:99 to 99:1 (e.g., 10:90, 20:80, 30:70, 40:60, 50:50, 60:40, 70:30, 80:20, 90:10). In some embodiments, the anti-neoplastic agent and immunosuppressant agent are present in a ratio by weight of approximately 1:1 (i.e., 50:50). In some embodiments, the anti-neoplastic agent and immunosuppressant agent are present in an amount ranging from about 0.1 μg/mm² to about 5 μg/mm² (e.g., 0.2, 0.4, 0.6, 0.8, 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, μg/mm²).

In some embodiments, coatings or coating compositions in accordance with the invention further include one or more anti-thrombotic agents, anti-proliferative agents, anti-inflammatory agents, anti-migratory agents, agents affecting extracellular matrix production and organization, anti-mitotic agents, anesthetic agents, anti-coagulant agents, vascular cell growth promoters, vascular cell growth inhibitors, cholesterol-lowering agents, vasodilating agents, and/or agents that interfere with endogenous vasoactive mechanisms.

In some embodiments, polymers suitable for the present invention contains a biodegradable polymer. In some embodiments, the biodegradable polymer is a polyester polymer. In some embodiments, suitable polyester polymer include, but are not limited to, poly(D,L-lactide-co-glycolide) (PLGA), polylactide (PLA), poly(L-lactide) (PLLA), poly(D,L-lactide (PDLA), polyglycolides (PGA), poly(D,L-glycolide) (PLG), and combinations thereof. In some embodiments, coatings in accordance with the invention further contain a calcium phosphate. In some embodiments, suitable calcium phosphates include, but are not limited to, amorphous calcium phosphate (ACP), dicalcium phosphate (DCP), tricalcium phosphate (TCP), pentacalcium hydroxyapatite (HAp), tetracalcium phosphate monoxide (TTCP), and combinations thereof. In some embodiments, the biodegradable polymer and calcium phosphate are present in a ratio (by weight) of about 1:99 to 99:1 (e.g., 10:90, 20:80, 30:70, 40:60, 50:50, 60:40, 70:30, 80:20, 90:10).

In some embodiments, polymers suitable for the present invention include a nonbiodegradable polymer. In some embodiments, suitable nonbiodegradable polymers include, but are not limited to, poly-n-butyl methacrylate (PBMA), polyethylene-co-vinyl acetate (PEVA), poly(styrene-b-isobutylene-b-styrene) (SIBS), and combinations thereof.

In some embodiments, the immunosuppressant agent and anti-neoplastic agent are present in the same layer. In some embodiments, the immunosuppressant agent and anti-neoplastic agent are present in different layers. In some embodiments, a cap layer is present over the layer containing the immunosuppressant agent and/or anti-neoplastic agent. In some embodiments, the cap layer contains a biodegradable polymer.

In another aspect, the present invention provides medical devices coated with coatings as described herein. In some embodiments, medical devices in accordance with the present invention include, but are not limited to, catheters, guide wires, balloons, filters, stents, stent grafts, vascular grafts, vascular patchs or shunts. In some embodiments, a medical device according to the invention is a stent. In some embodiments, a stent according to the invention is a metal stent (e.g., stents made of stainless steel, nitinol, tantalum, platinum, cobalt alloy, titanium, gold, a biocompatible metal alloy, iridium, silver, tungsten, or a combination thereof). In some embodiments, a stent according to the present invention is made from carbon, carbon fiber, cellulose acetate, cellulose nitrate, silicone, polyethylene teraphthalate, polyurethane, polyamide, polyester, polyorthoester, polyanhydride, polyether sulfone, polycarbonate, polypropylene, polyethylene, polytetrafluoroethylene, polylactic acid, polyglycolic acid, a polyanhydride, polycaprolactone, polyhydroxybutyrate, or a combination thereof.

In some embodiments, the present invention provides drug eluting systems including an implantable or insertable medical device and a coating or coating composition as described herein.

The present invention further provides methods of treating diseases, disorders, or conditions, in particular, those associated with hyperproliferation, using medical devices or drug eluting systems according to the invention. In some embodiments, the present invention provides methods of treating cardiovascular diseases using medical devices or drug eluting systems according to the invention (e.g., a stent coated with a coating of the invention).

In some embodiments, the present invention provides methods of treating restenosis by controlled release of sirolimus and paclitaxel from the surface of an implantable or insertable medical device (e.g., catheters, guide wires, balloons, filters, stents, stent grafts, vascular grafts, vascular patchs or shunts). In some embodiments, the present invention can be used to treat restenosis occurred in coronary arteries, peripheral arteries, brain arteries, kidney arteries, hepatic arteries, bile ducts, esophageal arteries and/or bronchial arteries, among others.

### BRIEF DESCRIPTION OF THE DRAWINGS

The drawings are for illustration purposes only, not for limitation.

FIG. 1. Illustration of an exemplary metal stent of the invention.

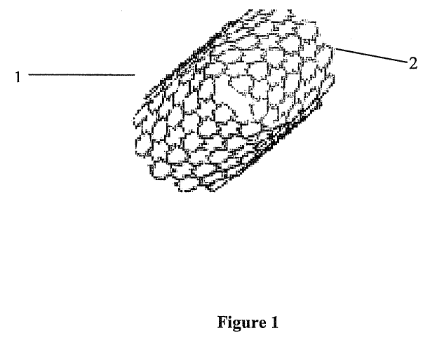

FIG. 2. Illustration of an exemplary multi-layer coating process.

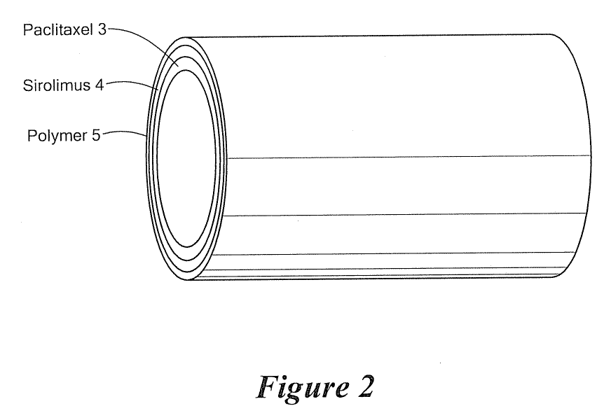

FIG. 3. Illustration of an exemplary pre-mixed coating process.

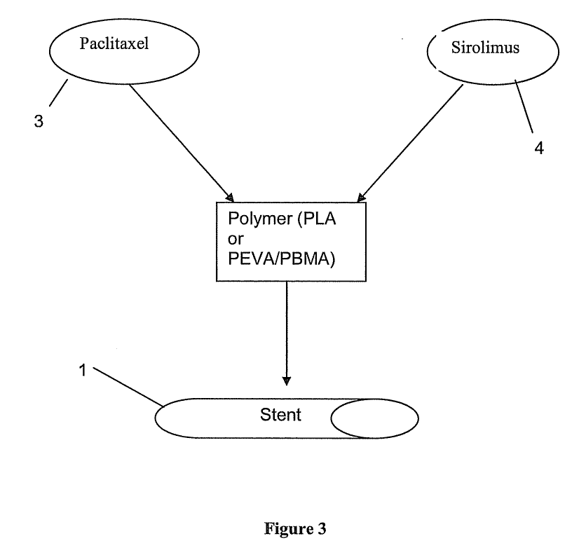

FIG. 4. An exemplary comparison of percentage of restenosis between stents coated with coatings containing a combination of sirolimus and paclitaxel and stents coated with coatings containing individual-drug alone at one month post implantation in rat carotid arteries.

FIG. 5. Exemplary plastic sections of rat carotid arteries implanted with four different drug coated stents at 28 days post implantation. Upper panel: Low-power (10×); Lower panel: Higher-power (40×) micrographs of the intima indicated by the respective boxes in the upper panel. The numbers in the lower panel are the measurement of neointima thickness. A: Polymer only; B: Sirolimus; C: Paclitaxel; D: Sirolimus and Paclitaxel combination coated stents. Note the significant difference of neointimal thickness among the four groups (both the upper and lower panels) and the inflammatory cell infiltration in polymer, sirolimus, and paclitaxel alone groups (lower panel of A, B and C). Also note the “healed” thin layer of neointima in Sirolimus and Paclitaxel combination coated stent (both upper and lower panels of D).

FIG. 6. Exemplary results illustrating an HPLC analysis of the residue level of sirolimus and paclitaxel in drug eluting stents coated with coatings containing sirolimus and paclitaxel combination at four weeks after elution. A: Pre-eluted composition drug coated DES at UV 218 nm; B: Four weeks post in vitro drug release. Both paclitaxel and sirolimus were continuously detected at 254 nm.

FIG. 7. An exemplary comparison of anti-restenosis effect between stents coated with coatings containing sirolimus and paclitaxel and stents coated with coatings containing individual drug in pig coronary arteries at four weeks post implantation.

FIG. 8. Exemplary histological comparison among pig coronary arteries at four weeks post implantation with stents coated with coatings containing sirolimus, paclitaxel, and a combination of sirolimus and paclitaxel. (A: Sirolimus; B: paclitaxel; C: sirolimus and paclitaxel combination). As shown in the picture, the rate of restenosis between the groups implanted with sirolimus and paclitaxel individually-coated stents is not significantly different. However, the rate of restenosis is significantly lower in the group implanted with stents coated coatings containing a combination of sirolimus and paciltaxol compared to the individually-coated stent groups.

FIG. 9. Illustration of a possible mechanism of how drug eluting stents coated with coatings containing a combination of sirolimus and paciltaxol effectively inhibit restenosis. Possible primary sites of action of sirolimus and paclitaxel are “G1” and “M” phases, respectively. However, a composition containing a combination of the two can block both “G1” and “M” phases. Due to the synergetic effect of two drugs, the dose of each drug in combination drug coatings may be significantly less than that in each individual drug coatings.

### DEFINITIONS

Agent: As used herein, the term “agent” refers to any substance that can be delivered to a tissue, cell, vessel, or subcellular locale. In some embodiments, the agent to be delivered is a biologically active agent (bioactive agent), i.e., it has activity in a biological system and/or organism. For instance, a substance that, when introduced to an organism, has a biological effect on that organism, is considered to be biologically active or bioactive. In some embodiments, an agent to be delivered is an agent that inhibit, reduce or delay cell proliferation.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Analogues or derivatives: As used herein, a derivative or an analogue refers to a compound can be formed from another compound. Typically, a derivative or an analogue of a compound is formed or can be formed by replacing at least one atom with another atom or a group of atoms. As used in connection with the present invention, a derivative or an analogue of a compound is a modified compound that shares one or more chemical characteristics or features that are responsible for the activity of the compound. In some embodiments, a derivative or an analogue of a compound has a pharmacophore structure of the compound as defined using standard methods known in the art. In some embodiments, a derivative or an analogue of a compound has a pharmacophore structure of the compound with at least one side chain or ring linked to the pharmacophore that is present in the original compound (e.g., a functional group). In some embodiments, a derivative or an analogue of a compound has a pharmacophore structure of the compound with side chains or rings linked to the pharmacophore substantially similar to those present in the original compound. As used herein, two chemical structures are considered “substantially similar” if they share at least 50% (e.g., at least 55%, at least 60%, at least 65%, at least 70%, at least 75%, at least 80%, at least 85%, at least 90%, at least 95%, at least 98%, or at least 99%) identical linkage bonds (e.g., rotatable linkage bonds). In some embodiments, two chemical structures are considered “substantially similar” if they share at least 50% (e.g., at least 55%, at least 60%, at least 65%, at least 70%, at least 75%, at least 80%, at least 85%, at least 90%, at least 95%, at least 98%, or at least 99%) identical atom coordinates defining the structures, or equivalent structures having a root mean square of deviation less than about 5.0 Å (e.g., less than about 4.5 Å, less than about 4.0 Å, less than about 3.5 Å, less than about 3.0 Å, less than about 2.5 Å, less than about 2.0 Å, less than about 1.5 Å, or less than about 1.0 Å). In some embodiments, two chemical structures are considered “substantially similar” if they share at least 50% (e.g., at least 55%, at least 60%, at least 65%, at least 70%, at least 75%, at least 80%, at least 85%, at least 90%, at least 95%, at least 98%, or at least 99%) identical atom coordinates defining surface-accessible features (e.g., hydrogen bond donors and acceptors, charged/ionizable groups, and/or hydrophobic patches), or equivalent features having a root mean square of deviation less than about 5.0 Å (e.g., less than about 4.5 Å, less than about 4.0 Å, less than about 3.5 Å, less than about 3.0 Å, less than about 2.5 Å, less than about 2.0 Å, less than about 1.5 Å, or less than about 1.0 Å).

Anti-neoplastic agent: As used herein, the term “anti-neoplastic agent” (also referred to as anti-proliferative agent) refers to an agent that inhibits and/or stops growth and/or proliferation of cells. An anti-neoplastic agent may display activity in vitro (e.g., when contacted with cells in culture), in vivo (e.g., when administered to a subject at risk of or suffering from hyperproliferation), or both. Exemplary anti-neoplastic agents include, but are not limited to, paclitaxel, enoxaprin, angiopeptin, carboplatin, vinorelbine, doxorubicin, gemcitabine, actinomycin-D, cisplatin, camptothecin, 5-fluorouracil, cyclophosphamide, 1-β-D-arabinofuranosylcytosine, or monoclonal antibodies capable of blocking smooth muscle cell proliferation, hirudin, and acetylsalicylic acid, amlodipine and doxazosin.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Combination therapy: The term “combination therapy”, as used herein, refers to those situations in which two or more different pharmaceutical agents are administered in overlapping regimens so that the subject is simultaneously exposed to both agents.

Control: As used herein, the term “control” has its art-understood meaning of being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparator.

Hyperproliferative condition: As used herein, the term “hyperproliferative condition” refers to undesirable cell growth. In some embodiments, hyperproliferative condition is associated with atherosclerosis, restenosis, proliferative vitreoretinopathy and psoriasis. The term is not intended to include cellular hyperproliferation associated with cancerous conditions. In some embodiments, undesirable cell growth refers to unregulated cell division associated with smooth muscle cells and/or fibroblasts. In some embodiments, undesirable cell growth is restenosis, which typically refers to the re-narrowing of opened artery after a surgical procedure such as stenting or PTCA procedure. Restenosis is typically due to a proliferative response of the intima, a layer of cells that line the lumen of the vessel, composed of connective tissue and smooth muscle cells (SMC).

Immunosuppressant agent: As used herein, the term “immunosuppressant agent” refers to any agent that reduces, inhibits or delays an immuno-reaction such as an inflammatory reaction. Exemplary immunosuppressants include, but are not limited to, sirolimus (RAPAMYCIN), tacrolimus, everolimus, dexamethasone, zotarolimus, tacrolimus, everolimus, biolimus, pimecrolimus, supralimus, temsirolimus, TAFA 93, invamycin and neuroimmunophilins.

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism such as a non-human animal.

Polymer: As used herein, the term “polymer” refers to any long-chain molecules containing small repeating units.

Prodrug: As used herein, the term “prodrug” refers to a pharmacological substance (drug) that is administered or delivered in an inactive (or significantly less active) form. Typically, once administered, the prodrug is metabolised in vivo into an active metabolite. The advantages of using prodrugs include better absorption, biocompatibility, distribution, metabolism, and excretion (ADME) optimization. Sometime, the use of a prodrug strategy increases the selectivity of the drug for its intended target.

Subject: As used herein, the term “subject” or “patient” refers to any organism to which systems, compositions or devices in accordance with the invention may be delivered or administered, e.g., for experimental, diagnostic, prophylactic, and/or therapeutic purposes. Typical subjects include animals (e.g., mammals such as mice, rats, rabbits, non-human primates, and humans; etc.).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of the disease, disorder, and/or condition.

Susceptible to: An individual who is “susceptible to” a disease, disorder, and/or condition has not been diagnosed with the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Sustained-release: As used herein, the term “sustained-release” refers to releasing (typically slowly) a drug over time. Typically, sustained-release formulations can keep steadier levels of the drug in the bloodstream. Typically, sustained-release coatings are formulated so that the bioactive agent is embedded in a matrix of polymers such that the dissolving agent has to find its way out through the holes in the matrix. In some embodiments, sustained-release coatings include several layers of polymers. In some embodiments, sustained-release coating matrix can physically swell up to form a gel, so that the drug has first to dissolve in matrix, then exit through the outer surface. As used herein, the terms of “sustained-release,” “extended-release,” “time-release” or “timed-release,” “controlled-release,” or “continuous-release” are used inter-changeably.

Therapeutically effective amount: As used herein, the terms “therapeutically effective amount” or “effective amount” of a therapeutic or bioactive agent refer to an amount that is sufficient, when administered to a subject suffering from or susceptible to a disease, disorder, and/or condition, to treat, diagnose, prevent, and/or delay the onset of the symptom(s) of the disease, disorder, and/or condition. In some embodiments, an effective amount refers to the amount necessary or sufficient to inhibit the undesirable cell growth. The effective amount can vary depending on factors know to those of skill in the art, such as the type of cell growth, the mode and the regimen of administration, the size of the subject, the severity of the cell growth, etc.

Therapeutic agent: As used herein, the phrase “therapeutic agent” refers to any agent that, when administered to a subject, has a therapeutic effect and/or elicits a desired biological and/or pharmacological effect.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition (e.g., hyperproliferation such as restenosis). Treatment may be administered to a subject who does not exhibit signs of a disease and/or exhibits only early signs of the disease for the purpose of decreasing the risk of developing pathology associated with the disease.

### DETAILED DESCRIPTION OF THE INVENTION

The present invention provides, among other things, coatings or coating compositions suitable for sustained drug delivery systems for the treatment of restenosis and other hyperproliferative diseases, disorders or conditions in vivo. In some embodiments, sustained drug delivery systems in accordance with the invention include an implantable or insertable medical device, a coating or coating composition that control sustained-release of bioactive agents that prevent, inhibit, reduce or delay the onset of restenosis.

### Restenosis

Restenosis, e.g., In-Stent Restenosis (ISR), formation is a multi-factorial, sequential process. For example, it is generally believed that three stages are involved in the ISR process: 1) Thrombotic Phase (day 0-3 after stent implantation). This phase is the initial response of artery tissue to stent implantation characterized with rapid activation, adhesion, aggregation and deposition of platelets and neutrophils to form a thrombus in the injured site. 2) Recruitment Phase. This phase occurs between day 3 to 8 characterized with an intensive inflammation cell infiltration. In this phase, the inflammation cells including leukocyte, monocytes, and macrophages were activated and infiltrated into the injured vessel wall. Subsequently, the recruited inflammation cells in the injured vessel wall provide the key stimulus for subsequent smooth muscle cell (SMC) proliferation and migration. In addition, the release and expression of adhesion cells, cytokines, chemokines, and growth factors by platelets, monocytes, and SMCs contribute to the further recruitment, infiltration at the site of injury, and further proliferation/migration of SMCs from media to neointima in the days after injuries. Anti-inflammation drugs (e.g., dexamethasone) and immunosuppressant drugs (e.g., sirolimus) are thought to delay or inhibit this phase. 3) Proliferate Phase. This phase last 1 to 3 months depending on the thickness of the residual thrombus and the rate of growth. At this stage, inflammation cells colonize the residual thrombus, forming a “cap” across the mural thrombus. The cells progressively proliferate, resorbing residual thrombus until all thrombus is gone and is replaced by the neointima tissue. These processes are induced by the early-phase events and also the exposure to circulatory mitogens (e.g., angiotensin II, plasmin). Vascular SMCs, otherwise in the quiescent phase of the cell cycle, are now triggered by early gene expression to undergo proliferation and migration with subsequent synthesis of extra cellular matrix and collagen, resulting in neointima formation. The process of neointimal growth, which consists of SMC, extracellular matrix, and macrophages recruited over a period of several weeks, is similar to the process of tumor tissue growth. This pathologic similarity between tumor cell growth and benign neointimal formation has led to the discovery of anti-tumor drugs as effective agents for the treatment of ISR.

### Sustained Drug Delivery Systems

A typical drug delivery system (also referred to as drug eluting system) for treating, preventing, inhibiting, or delaying the onset of retenosis include an implantable or insertable medical device (e.g., stent), coating or coating matrix, and bioactive agents. Implantable or insertable medical devices such as a stent provide a basic platform to deliver sufficient drug to the diseased arteries. Coating or coating matrix provides a reservoir for sustained delivery of bioactive agents. Typically, achieving compatibility between the implantable or insertable medical device, coating matrix, drugs and vessel wall is central for successful development of a drug delivery system.

Implantable or Insertable Medical Devices

A typical platform for delivery of anti-restenosis drugs to an diseased arterial wall is an implantable or insertable medical device. A desirable drug-delivery platform typically has a larger surface area, minimal gaps between endothelial cells so as to minimize plaque prolapsed (displacement) in areas of large plaque burden, and minimal deformation (adaptation in shape or form) after implantation. Exemplary implantable or insertable medical devices suitable for the present invention include, but are not limited to, catheters, guide wires, balloons, filters, stents, stent grafts, vascular grafts, vascular patchs or shunts.

In some embodiments, medical devices suitable for the invention are stents. Stents suitable for the present invention include any stent for medical purposes, which are known to the skilled artisans. Exemplary stents include, but are not limited to, vascular stents such as self-expanding stents and balloon expandable stents. Examples of self-expanding stents useful in the present invention are illustrated in U.S. Pat. Nos. 4,655,771 and 4,954,126 issued to Wallsten and U.S. Pat. No. 5,061,275 issued to Wallsten et al. Examples of appropriate balloon-expandable stents are shown in U.S. Pat. No. 5,449,373 issued to Pinchasik et al.

Suitable stents can be metal or non-metal stents. Exemplary biocompatible non-toxic mental stents include, but not limited to, stents made of stainless steel, nitinol, tantalum, platinum, cobalt alloy, titanium, gold, a biocompatible metal alloy, iridium, silver, tungsten, or combinations thereof. Exemplary biocompatible non-metal stents include, but not limited to, stents made from carbon, carbon fiber, cellulose acetate, cellulose nitrate, silicone, polyethylene teraphthalate, polyurethane, polyamide, polyester, polyorthoester, polyanhydride, polyether sulfone, polycarbonate, polypropylene, polyethylene, polytetrafluoroethylene, polylactic acid, polyglycolic acid, a polyanhydride, polycaprolactone, polyhydroxybutyrate, or combinations thereof. Other polymers suitable for non-metal stents are shape-memory polymers, as described for example by Froix, U.S. Pat. No. 5,163,952, which is incorporated by reference herein. Stents formed of shape-memory polymers, which include methacylate-containg and acrylate-containing polymers, readily expand to assume a memory condition to expand and press against the lumen walls of a target vessel, as described by Phan, U.S. Pat. No. 5,603,722, which is incorporated by reference in its entirety.

Typically, implantable or insertable medical devices are adapted to serve as a structural support to carry a polymer based coating as described herein. For example, a polymer-based, drug containing fiber can be threaded through a metal stent aperture. The metal stent typically provides the mechanical support in the vessel after deployment for maintaining vessel patency, and the polymer thread provides a controlled release of bioactive agents. Another example is a drug-loaded polymer sheath encompassing a stent, as described in U.S. Pat. No. 5,383,928 (Scott, et al). Yet another example is a polymer stent which coexpand with a metal stent when placed in the target vessel, as described in U.S. Pat. No. 5,674,242 (Pham, et al).

Coatings or Coating Compositions

Coatings (also referred to as coating matrix, or coating compositions) are an important component of the drug-eluting system. Through coating, drugs are retained during deployment and drug-eluting releasing kinetics is modulated. Typically, a coating in accordance with the present invention is formulated to contain one or more polymers and at least one bioactive agent such that the coating controls sustained-release of the bioactive agent. Typically, the one or more polymers form a matrix or several layers of matrixes to embed a bioactive agent. In some embodiments, a sustained-release coating may form a gel that can physically swell after implantation into a blood vessel. In some embodiments, a coating of the invention is formulated to control sustained release of a bioactive agent for at least up to 4 weeks (e.g., 5 weeks, 6 weeks, 7 weeks, 8 week, 9 weeks, 10 weeks, 11 weeks, 12 weeks, or more). The duration of the sustained-release can be measured by various methods known in the art or as described in the Examples section.

Coating substances suitable for the invention typically maintain their physicochemical characteristics after sterilization and, after expansion (for example, stent expansion), be capable of being stretched without flaking or delaminating from the surface. A coating matrix can be attached to the surface of an implantable or insertable medical device by either covalent bonds (e.g., C—C bonds, sulfur bridges) or non-covalent bonds (e.g., ionic, hydrogen bonds).

Coating compositions that are useful for the present invention may be a solution or a suspension comprising one or more bioactive agents, polymeric materials and solvent, or may be solid comprising one or more bioactive agents, polymeric materials. Components for a coating composition can be pre-blended and then coated on the surface of a medical device. Alternatively, individual components can be applied layer-by-layer onto the surface of a medical device. Suitable coating technologies include, but are not limited to, dipping, spraying, brushing, and vaporing deposition etc.

### 1. Polymers

Polymers suitable for the coatings of the present invention include any polymers that are biologically inert and not induce further inflammation (e.g., biocompatible and avoids irritation to body tissue). In some embodiments, suitable polymers are non-biodegradable. Exemplary non-biodegradable polymers include, but are not limited to, poly-n-butyl methacrylate (PBMA), polyethylene-co-vinyl acetate (PEVA), poly(styrene-b-isobutylene-b-styrene (SIBS), and combinations or analogues thereof.

Other non-biodegradable polymers that are suitable for use in this invention include polymers such as polyurethane, silicones, polyesters, polyolefins, polyamides, polycaprolactam, polyimide, polyvinyl chloride, polyvinyl methyl ether, polyvinyl alcohol, acrylic polymers and copolymers, polyacrylonitrile, polystyrene copolymers of vinyl monomers with olefins (such as styrene acrylonitrile copolymers, ethylene methyl methacrylate copolymers, ethylene vinyl acetate), polyethers, rayons, cellulosics (such as cellulose acetate, cellulose nitrate, cellulose propionate, etc.), parylene and derivatives thereof; and mixtures and copolymers of the foregoing.

In some embodiments, suitable polymers are biodegradable. In some embodiments, a suitable biodegradable polymer is a polyester. Exemplary polyester polymers suitable for the invention include, but are not limited to, poly(D,L-lactide-co-glycolide) (PLGA), polylactides (PLA), Poly(L-lactide) (PLLA), Poly (D,L-lactide) (PDLA), polyglycolides (PGA), and combinations or analogues thereof. PLA and PGA are desirable for medical applications because they have lactic acid and glycolic acid as their degradation products, respectively. These natural metabolites are ultimately converted to water and carbon dioxide through the action of enzymes in the tricarboxylic acid cycle and are excreted via the respiratory system. In addition, PGA is also partly broken down through the activity of esterases and excreted in the urine. Along with its superior hydrophobicity, PLA is more resistant to hydrolytic attack than PGA, making an increase of the PLA:PGA ratio in a PLGA copolymer result in delayed degradability.

Thus, although the invention can be practiced by using a single type of polymer to form the coating layer(s), it is desirable to use various combinations of polymers. The appropriate mixture of polymers can be coordinated with biologically active materials of interest to produce desired effects when coated on a medical device in accordance with the invention.

In some embodiments, polymers suitable for the invention include calcium phosphates. In some embodiments, calcium phosphates are used in combination with biodegradable polymers. Without wishing to be bound to a particular theory, it is believed that combining calcium phosphate material with biodegradable polymers may buffer the acidic materials released by biodegradation, and therefore provide coating that will induce less inflammation. In some embodiments, the ratio of the polyester polymer and the calcium phosphate ranges from about 99:1 to 1:99 (e.g., 10:90, 20:80, 30:70, 40:60, 50:50, 60:40, 70:30, 80:20, 90:10).

Exemplary calcium phosphates that may be used in the current invention include, but not limited to, amorphous calcium phosphate (ACP), dicalcium phosphate (DCP), tricalcium phosphate (TCP), pentacalcium hydroxyl Apatite (HAp), tetracalcium phosphate monoxide (TTCP) and combinations or analogues thereof.

For example, ACP is an important intermediate product for in vitro and in vivo apatite formation with high solubility and better biodegradability. It was mainly used in the form of particles or powders, as an inorganic component incorporated into biopolymers, to adjust the mechanical properties, biodegradability, and bioactivity of the resulting composites. Based on the similarity of ACP to the inorganic component of the bone, ACP is particular useful as a bioactive additive in medical devices to improve remineralization. Based on its solubility, coatings containing ACP may release ions into aqueous media, forming a favorable super saturation level of Ca²⁺ and PO₄³− ions for the formation of apatite. The ion release may neutralize the acidity resulted from polymer biodegradation, retarding bioresorptive rate and eliminating inflammation occurrence.

### 2. Bioactive Agents

The current invention provides coatings or coating compositions containing at least an anti-neoplastic agent and/or an immunosuppressant agent. In some embodiments, an anti-neoplastic agent suitable for the invention is paclitaxel, or a prodrug or analog thereof. In some embodiments, anti-neoplastic agents suitable for the invention is selected from carboplatin, vinorelbine, doxorubicin, gemcitabine, actinomycin-D, cisplatin, camptothecin, 5-fluorouracil, cyclophosphamide, 1-β-D-arabinofuranosylcytosine, or a combination or analogs thereof. In some embodiments, an immunosuppressant agent suitable for the invention is sirolimus, or a prodrug or analog thereof. In some embodiments, immunosuppressant agents suitable for the invention is selected from zotarolimus, tacrolimus, everolimus, biolimus, pimecrolimus, supralimus, temsirolimus, TAFA 93, invamycin or neuroimmunophilins, or a combination or analogs thereof.

Paclitaxel, an extract from the bark of the Pacific yew tree Taxus brevifolia. The anti-proliferative activity of paclitaxel is a result of concentration-dependent and reversible binding to microtubules, specifically to the β-subunit of tubulin at the N-terminal domain. This binding promotes polymerization of tubulin to form stable microtubules by reducing the critical concentration of tubulin required for polymerization and preventing depolymerization of the microtubules; the structure of the microtubules is stabilized by the formation of bundles and multiple asters.

Paclitaxel produces distinct dose-dependent effects within the cell: at low doses it causes G1 arrest during interphase by inducing p53 and p21 tumor suppression genes, resulting in cytostasis. At high doses, the drug are thought to affect the G₂-M phase of the cell cycle. Since the microtubules must be disassembled for transition from the G2 to the M phase to take place, and paclitaxel stabilizes the microtubule structure, mitotic arrest occurs in the presence of paclitaxel. Alternatively, high doses may affect the M-G₁ phase causing post-mitotic arrest and possibly apoptosis. In addition to these actions, activation of some protein kinases and serine protein phosphorylation are associated with depolymerization of microtubules, and are therefore inhibited by paclitaxel. Thus, any paclitaxel analogs that retain or improve the cell cycle inhibitory function of paclitaxel as described herein can be used in accordance with the invention.

Sirolimus (rapamycin), a natural macrolide antibiotic with potent immunosuppressant properties, was first approved by the FDA in 1999 for use as an anti-rejection agent following organ transplantation. Its use in intracoronary stenting was based on the premise that the anti-proliferative properties of the drug would inhibit the neointimal hyperplasia (NIH) associated with restenosis following stent implantation. An important mechanism of Sirolimus action is entry into target cells and binding to the cytosolic immunophilin FK-binding protein-12 (FKBP-12) to form a Sirolimus:FKBP-12 complex that interrupts signal transduction, selectively interfering with protein synthesis. After binding with FK-binding protein-12 (FKBP-12), Sirolimus inhibits the activity of the mammalian target of Rapamycin (mTOR) and eventually the activity of the cyclin-dependent kinase (cdk)/cyclin complexes, as well as the phosphorylation of retinoblastoma protein, thereby preventing advancement of the cell cycle from G1 to S phase. Thus, any Sirolimus analogs that retain or improve the cell cycle inhibitory function of Sirolimus as described herein can be used in accordance with the invention.

In preferred embodiments, the present invention provides coatings or coating compositions containing a combination of an anti-neoplastic agent (such as paclitaxel or its prodrug or anologs) and an immunosuppressant agent (such as sirolimus or its prodrug or anologs).

Several combination therapies have been investigated previously in the treatment of in-stent restenosis. However, all those investigations involved the combination of anti-plastic (Paclitaxol) or immunosuppressant drug (Sirolimus) with anti-thrombotic agents such as Glycoprotein IIB/IIIA inhibitor or heparin) (Leon MB and Bakhai Ameet, “Drug releasing stent and glycoprotein IIb/IIIA inhibitor: combination therapy for the future,” Am Heart J 2003; 146:S13-7) or nitric oxide (Lin-Chiaen, and Delano Yang et al. “Combination of paclitaxel and nitric oxide as a novel treatment for the reduction of restenosis,” J. Med. Chem. 2004; 47: 2276-2282). The purpose of adding anti-thrombotic drugs to coated stent is to prevent thrombosis. However, the efficacies of these combinations in inhibition of neointimal hyperplasia after stent implantation are limited. The one possible reason for the limited effects of these combinations is the physiochemical incompatibility among combined drugs. Local drugs that are retained within the blood vessel are more effective than those are not. Both heparin and nitric oxide compounds are so soluble and diffusible that they simply cannot stay in the artery for more than a few minutes after release. US patent application to Hsu Li-Chien (US-2004/0037886: Drug Eluting Stent for Medical Implant) had disclosed a modified coating system to increase the compatibility among combined drugs (hydrophilic and hydrophobic drugs). However, as discussed below, the combination used in the modified coating system in Hsu's patent application is completely different from the combination therapies contemplated in the present application.

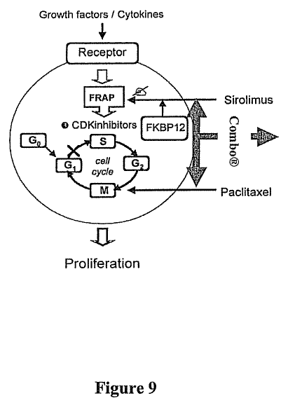

Coatings of the present invention are developed to harness synergistic effects between an anti-neoplastic agent and an immunosuppressant agent. For example, contrary to the above-described hydrophilic and hydrophobic drug combinations, both sirolimus and paclitaxel are hydrophobic, and retained well in blood vessel wall for up to three days through specifically binding to their individual binding proteins (Levin, A. D. et al., “Edelman Specific binding to intracellular proteins determines arterial transport properties for rapamycin and paclitaxel,” PNAS 2004; 101(25):9463-67) after releasing from stent. Therefore, it is contemplated that a combination of these two drugs in a coating according to the invention may work synergistically to inhibit restenosis including neointimal hyperplasia. Medical devices coated with a combination of bioactive agents would require lower doses of each agent to achieve the same or even greater anti-restenosis effects with less side-effects compared to otherwise identical medical devices coated with individual agent alone. FIG. 9 depict a possible synergistic mechanism in accordance with the invention.

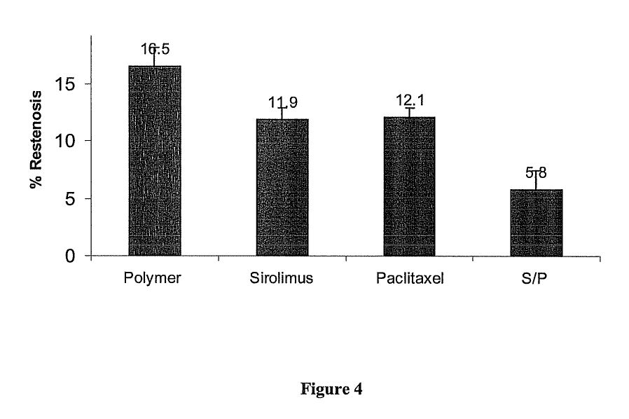

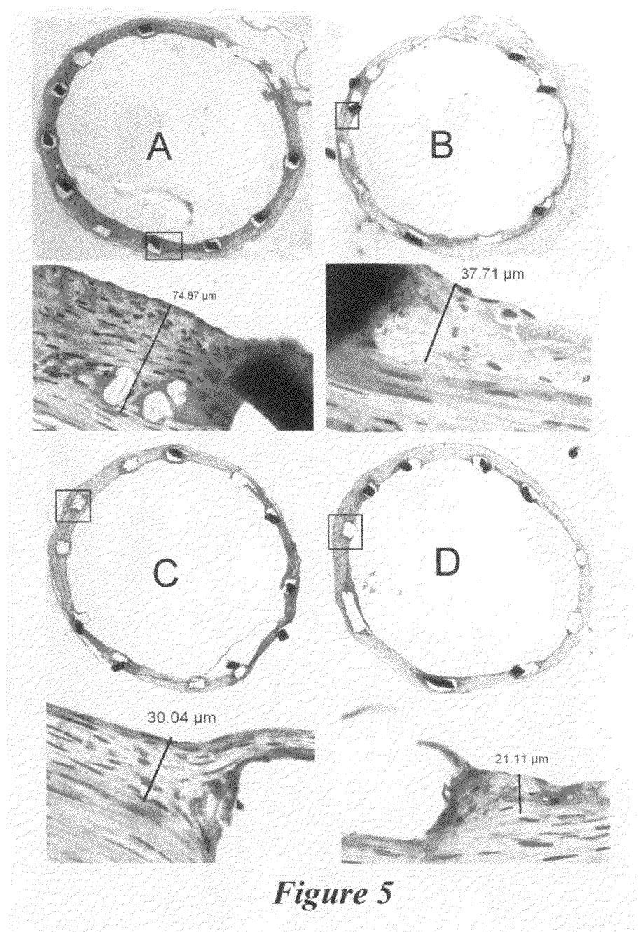

Indeed, the present inventors have demonstrated that sirolimus and paclitaxel do act synergistically in a stent coating in inhibiting restenosis. In fact, as described in the Examples section, stents coated with a coating containing a combination of sirolimus and paclitaxel are surprisingly effective in inhibiting, preventing, and/or delaying the onset of restenosis in vivo. For example, stents coated with both sirolimus and paclitaxel were approximately 50% more effective in reducing restenosis in rat carotid arteries than stents coated with paclitaxel or sirolimus alone (FIGS. 4 and 5). In addition, the rate of restenosis in porcine coronary arteries implanted with stents coated with a combination of sirolimus and paclitaxel is significantly less (6.7%) compared to stents coated with either sirolimus or paciltaxol alone (14.5% and 15.6%, respectively). As shown in FIG. 5, stents coated with a combination of sirolimus and paclitaxel (D) also has the least neointima formation among three groups. The inner wall of arteries implanted stents coated with a combination of sirolimus and paclitaxel was covered by a thin layer of endothelial cells, which is a strong indication of the reendothelialization process taking place. Therefore, the present inventors have demonstrated that coatings containing a combination of anti-neoplastic agents and an immunosuppressant agents in accordance with the invention promote significantly less restenosis formation in vivo. Therefore, the present invention provides new and powerful drug eluting systems (e.g., drug eluting stents) for treatment of restenosis (e.g, arterial restenosis).

Bioactive agents suitable for the invention may also include anti-thrombogenic agents such as heparin, heparin derivatives, urokinase, and PPack (dextrophenylalanine proline arginine chloromethylketone); anti-inflammatory agents such as glucocorticoids, betamethasone, dexamethasone, prednisolone, corticosterone, budesonide, estrogen, sulfasalazine, and mesalamine; other antineoplastic/antiproliferative/anti-miotic agents such as 5-fluorouracil, cisplatin, vinblastine, vincristine, epothilones, methotrexate, azathioprine, halofuginone, adriamycin, actinomycin and mutamycin; endostatin, angiostatin and thymidine kinase inhibitors, and its analogs or derivatives; anesthetic agents such as lidocaine, bupivacaine, and ropivacaine; anti-coagulants such as D-Phe-Pro-Arg chloromethyl keton, an RGD peptide-containing compound, heparin, antithrombin compounds, platelet receptor antagonists, anti-thrombin anticodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors and tick antiplatelet peptides; vascular cell growth promotors such as growth factors, Vascular Endothelial Growth Factors (FEGF, all types including VEGF-2), growth factor receptors, transcriptional activators, and translational promotors; vascular cell growth inhibitors such as antiproliferative agents, growth factor inhibitors, growth factor receptor antagonists, transcriptional repressors, translational repressors, replication inhibitors, inhibitory antibodies, antibodies directed against growth factors, bifunctional molecules including a growth factor and a cytotoxin, bifunctional molecules including an antibody and a cytotoxin; cholesterol-lowering agents; vasodilating agents; and agents which interfere with endogenous vasoactive mechanisms; anti-oxidants, such as probucol; antibiotic agents, such as penicillin, cefoxitin, oxacillin, tobranycin angiogenic substances, such as acidic and basic fibrobrast growth factors, estrogen including estradiol (E2), estriol (E3) and 17-Beta Estradiol; and drugs for heart failure, such as digoxin, beta-blockers, angiotensin-converting enzyme (ACE) inhibitors including captopril and enalopril.

In addition, bioactive agents suitable for the present invention include nitric oxide adducts, which prevent and/or treat adverse effects associated with use of a medical device in a patient, such as restenosis and damaged blood vessel surface. Typical nitric oxide adducts include, but are not limited to, nitroglycerin, sodium nitroprusside, S-nitroso-proteins, S-nitroso-thiols, long carbon-chain lipophilic S-nitrosothiols, S-nitrosodithiols, iron-nitrosyl compounds, thionitrates, thionitrites, sydnonimines, furoxans, organic nitrates, and nitrosated amino acids, preferably mono- or poly-nitrosylated proteins, particularly polynitrosated albumin or polymers or aggregates thereof. The albumin is preferably human or bovine, including humanized bovine serum albumin. Such nitric oxide adducts are disclosed in U.S. Pat. No. 6,087,479 to Stamler et al. which is incorporated herein by reference.

Bioactive agents may be encapsulated in micro or nano-capsules by the known methods.

Bioactive agents can be used with (a) biologically non-active material(s) including a carrier or an excipient, such as sucrose acetate isobutyrate (SABER™ commercially available from SBS) ethanol, n-methyl pymolidone, dimethyl sulfoxide, benzyl benxoate, benzyl acetate, albumine, carbohydrate, and polysacharide. Also, nanoparticles of the biologically active materials and non-active materials are useful for the coating formulation of the present invention.

Bioactive agents including anti-neoplastic agents and immunosuppressant agents may be present in one single layer. Alternatively, individual agents (such as anti-neoplastic agents and immunosuppressant agents) may be present in separate layers. In some embodiments, a drug-free polymer layer (also referred to as cap layer) can be coated over a layer or layers containing an anti-neoplastic agent and/or immunosuppressant agent to act as a diffusion barrier.

Additional compositions combining anti-neoplastic agents and an immunosuppressant agents such as sirolimus and paclitaxel or prodrugs or analogs thereof, are described in the U.S. application Ser. No. 11/144,917. Additional coating formulations containing anti-neoplastic agents and an immunosuppressant agents such as sirolimus and paclitaxel or prodrugs or analogs thereof, and biodegradable polymers are described in U.S. patent application Ser. No. 11/843,528. The disclosures of U.S. application Ser. Nos. 11/144,917 and 11/843,528 are hereby incorporated by references.

### Therapeutic Applications

Inventive drug eluting or delivery systems described herein can be used to inhibit, reduce, delay, or eliminate the formation of restenosis or other undesirable hyperproliferative conditions. In some embodiments, the present invention can be used to treat restenosis occurred in coronary arteries, peripheral arteries, brain arteries, kidney arteries, hepatic arteries, bile ducts, esophageal arteries and/or bronchial arteries, among others. In some embodiments, drug eluting systems according to the invention can be used to treat diseases, disorders or conditions associated with hyperproliferation or undesirable cell growth.

For example, inventive drug eluting systems according to the invention can be used to decrease the thickness of intimas that result from smooth muscle proliferation following angioplasty, either in an animal model or in human. In some embodiments, inventive drug eluting systems according to the invention are used to delay onset of visible intima hyperplasia, for example, as observed histologically or by angiographic techniques, following angioplasty. In some embodiments, inventive drug eluting systems according to the invention are used to eliminate (e.g., substantially reduce and/or delay the onset of) intimal hyperplasia in a blood vessel such that sufficient blood flow in the vessel is established and further surgical intervention is not necessary.

In some embodiments, drug eluting systems of the invention include coated drug delivery catheters. Drug delivery catheters are described in U.S. Pat. Nos. 5,558,642, 5,295,962, 5,171,217 and 5,674,192. Typically, such catheters have a flexible shaft and an inflatable balloon at the distal end of the shaft. The catheter is inserted into a vessel in an un-inflated condition and the balloon member is inflated and apertures in the balloon assembly provide drug carried in the catheter to be delivered to the target site. The drugs can be carried in solution form, entrapped in microparticles of a physiologically compatible polymer or incorporated into a polymer, such as a hydrogel, which is coated on the balloon region for rapid release of the drug during expansion of the balloon. Such catheters can be used in the conjunction with a balloon angioplasty procedure.

In some embodiments, drug eluting systems according to the invention include coated infusion catheters or drug delivery guidewires. An infusion catheter provides delivery of agents to a target site by placing the tip of the catheter at the site and connecting the catheter to a pump. The tip of the catheter generally includes opening through which the agent is pumped at desired rate to the target site (U.S. Pat. No. 5,720,720). A drug delivery guidewire has been described in the U.S. Pat. No. 5,569,197, which the guidewire is hollow and has an opening at its distal end for infusion of a drug therethrough.

In preferred embodiments, drug eluting systems according to the invention include coated stents (also referred to as drug eluting stents). For example, endovascular stent for use following balloon angioplasty are known in the art and described in, for example, U.S. Pat. No. 5,395,390(Simon), U.S. Pat. No. 4,739,762 (Palmaz), U.S. Pat. No. 5,195,984 (Schayz) and U.S. Pat. No. 5,163,952 (Froix). In some embodiments, suitable stents are metal stents. Exemplary biocompatible and nontoxic metals suitable for coated stents include nickel-titanium alloys, tantalum, and steel. In some embodiments, coating compositions can be absorbed onto a stent or incorporated into indentations, e.g., pockets, grooves or pits, formed on the surface of the stent. In some embodiments, stents are coated by dipping the stents into coating solutions according to the invention or spraying coating solutions onto the surface of the stents.

In some embodiments, compositions according to the invention can also be administered by any route which provides effective therapy for the inhibition of restenosis or other hyperproliferative conditions. Suitable administration routes include any routes of administration which allow the compositions to perform its intended function of inhibiting undesirable cell growth. Such routes include but not limited to systemic administration including bolus, pulsed, and continuous injection intravenously, subcutaneously, intramuscularly, intraperitoneally, etc.

### EXAMPLES

### Example 1

### Pre-Mixed Coating of Composition in PBMA/PEVA Co-Polymer on Metal Stents

Forty metal stents were dip-coated with four different drug-polymer formulations: Polymer only, Sirolimus only, Paclitaxel only, and Sirolimus/Paclitaxel combination. Each group had 10 stents. The coating polymer was composed of 50% PEVA and 50% PBMA which was made by dissolving PEVA (1.75 mg) and PBMA (1.75 mg) in tetrahydrofuran (THF, 1 mL) (Sigma, Saint Louis, Mo.). The coating solutions for Sirolimus only and Paclitaxel only groups were made by dissolving 5 mg of either Sirolimus or Paclitaxel into 1 mL copolymer solutions. The Sirolimus/Paclitaxel combined formulation comprises 2.5 mg (half dose) of Sirolimus and 2.5 mg (half dose) of Paclitaxel in 1 mL copolymer solution. The drug and polymer concentrations in the above coating solution were optimized from several preliminary tests. Prior to coating, all stents were weighed and cleaned with an ultrasonic cleaner at 37° C. for 30 minutes. Stents were dip-coated in the solution for three seconds and then slowly removed from coating solution. After air-drying completely at room temperature, the stents were weighed again in order to calculate the total amount of coated drug/polymer. The total loading amount of the drugs can then be calculated from the total coated weight according to their ratio in the solution. Table 1 summarizes the study. As shown in the table, a single dip-coating process can achieve approximately 1 μg/mm² (stent surface area) of drugs among all three drug-coated groups which is comparable to both commercially available Sirolimus coated CYPHER™ stent and Paclitaxel coated TAXUS™ stent.

TABLE 1
Summary of Pre-mixed Drug Coating of Sirolimus and Paclitaxel Stents

| Groups | Co-polymer | Sirolimus | Paclitaxel | Combination |
|---|---|---|---|---|
| Formulations | 1.75 mg PEVA, | 1.75 mg PEVA, | 1.75 mg PEVA, | 1.75 mg PEVA, |
| | 1.75 mg PBMA in | 1.75 mg PBMA and 5 mg | 1.75 mg PBMA and 5 mg | 1.75 mg PBMA, 2.5 mg |
| | 1 ml THF | Sirolimus in 1 ml THF | Paclitaxel in 1 ml THF | Paclitaxel and 2.5 mg |
| | | | | Sirolimus, 1 ml THF |
| Number of Stent | 10 | 10 | 10 | 10 |
| Total Coated Weight (ug/stent) | 10 ± 1.9 | 21 ± 2.7 | 24 ± 3.6 | 21 ± 3.1 |
| Total Coated Drug (ug/stent) | 0 | 13 ± 1.6 | 15 ± 2.1 | 6.5 ± 0.9/6.5 ± 0.9 |
| ug/mm² (stent surface) | | 0.95 ± 0.1 | 1 + 0.1 | 0.48 ± 0.1/0.48 ± 0.1 |

PBMA: Poly-n-Butyl Methacrylate;
PEVA: Polyethylene-vinyl Acetate;
THF: Tetrahydrofutan

### Example 2

### Multi-Layered Coating of the Composition in PLA Polymer

PLA Poly(lactic acid) was dissolved in chloroform at a concentration of 6.7 mg/ml. Two aliquots of 2 mL each were the solution into two parts, each part has 2 ml. Sirolimus (5 mg) was added to the PLA/CHCl₃ solution (2 mL) to give a sirolimus-polymer solution (2.5 mg/ml). Paclitaxel (5 mg) was added to the PLA/CHCl₃ solution (2 mL) to give a paclitaxel-polymer solution (2.5 mg/ml). To make combination coated stents, the bare metal stents were dipped into the Sirolimus coating solution for thirty seconds to make the first layer, and then air-dried completely. The stent was then dipped into the Paclitaxel coating solution for another thirty seconds and air-dried completely at room temperature. The stents were weighed to calculate the total amount of coated drug/polymer. Table 2 is a summarization of total amount of drugs coated on the stent by this method.

TABLE 2
Multi-layer Coating of Sirolimus
and Paclitaxel Combination Stents

| Formulation | PLA polymer | Sirolimus | Paclitaxel | Combination |
|---|---|---|---|---|
| No# Stents | 10 | 10 | 10 | 10 |
| Drug coated (ug) | 10 | 14 | 15 | 7.1/7.1 |
| μg/mm2 | | 0.96 | 1.01 | 0.51/0.51 |

### Example 3

### HPLC Analysis of Sirolimus and Paclitaxel Drug Releasing in the Coated Stent in In-Vitro

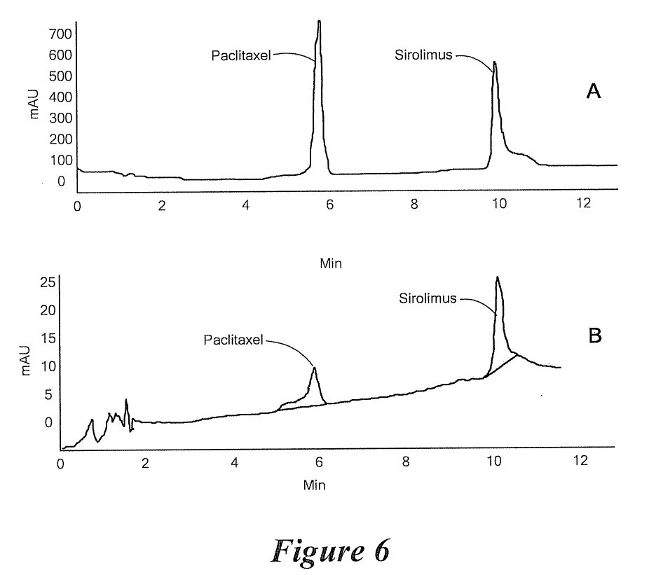

To determine the stability of Sirolimus and Paclitaxel in the combined formulation, an in vitro drug eluting study was performed in three stents coated with the combined formulation as described in the Example 1. Each stent was placed in a 2.5 mL PBS solution contained in a 10 mL culture tube with a screw cap. The tube was consistently shaken in a water bath at 200 RPM at 37° C. The PBS solution was changed daily to keep the stent in a fresh condition. The drug releasing process was stopped at 1, 2, and 4 weeks following the shaking with one stent in each time point. The stent was then placed in a 1 mL extracting solution (100% ethanol) and continuously shaken at room temperature overnight. The 10 μL extracted solutions which contain the remaining Sirolimus and Paclitaxel after eluting were further analyzed by HPLC (HP16 series 1090, Hewlett-Packard Co. Palo Alto, Calif.). The samples were analyzed on a C18-reverse phase column (HP: 4.6×100 mm RP18) using a mobile phase consisting of 0.005% TFA buffer (0.05 ml Trifluoroacetic acid in 1000 ml acetonitrile) delivered at a flow rate of 1.0 mL/min. In all three samples (at 1, 2 and 4 weeks), both Paclitaxel and Sirolimus peaks were detected by UV between 218 nm and 280 nm. FIG. 6 depicts the HPLC analysis of Sirolimus and Paclitaxel in composition coated drug eluting stents at four weeks after elution.

### Example 4

### Anti-Restenosis Effect of Composition Coated Stent in Rat Carotid Arteries

To compare inhibition of in-stent restenosis with the Sirolimus/Paclitaxel combined coating versus coatings containing sirolimus or paclitaxel alone in vivo, twelve stents coated with either Co-polymer only (3 stents), Sirolimus only (3 stents), Paclitaxel only (3 stents) and Sirolimus/Paclitaxel combination (3 stents) as described in Example 1 were implanted into twelve Sprague Dawley (SD) rat carotid arteries. At 4 weeks post implantation, all experimental rats were sacrificed with overdose of Ketamine and Xylazine intraperitioneally. The implanted carotid arteries were carefully isolated, removed, plastic-embedded and analyzed morphometrically and histopathologically.

FIGS. 4 and 5 depict the differences in the rates of in-stent restenosis among four experimental groups at four weeks post-stenting. Compared to polymer group, both sirolimus and paclitaxel coated stents can significantly reduce the rate of in-stent restenosis (11.9% and 12.1% vs. 16.5% respectively, P<0.05), but there are no significance difference between Paclitaxel and Sirolimus alone coated groups (11.9% vs. 12.1%, p>0.05), which correlates well with most recently published clinical trial data of both Sirolimus and Paclitaxel coated stents. Inventive composition coated stents further lower the rate of in-stent restenosis to a level of 5.8%, which is approximately 50% reduction of restenosis comparing to coated stents containing either sirolimus or paclitaxel alone.

### Example 5

### Anti-Restenosis Effects of Composition Coated Stents in Porcine Coronary Arterial Implantation

To further confirm the finding in Example 4 that composition coated stents are more effective at inhibiting restenosis than stents coated with either sirolimus or paclitaxel alone, nigh stents (three sirolimus-only stents, three paclitaxol only stents, and three composition coated stents) were implanted into domestic pig coronary arteries. At 4 weeks post implantation, all pigs were euthanized and the stented coronary arteries were carefully isolated, removed, plastic-embedded and analyzed morphometrically and histopathologically.

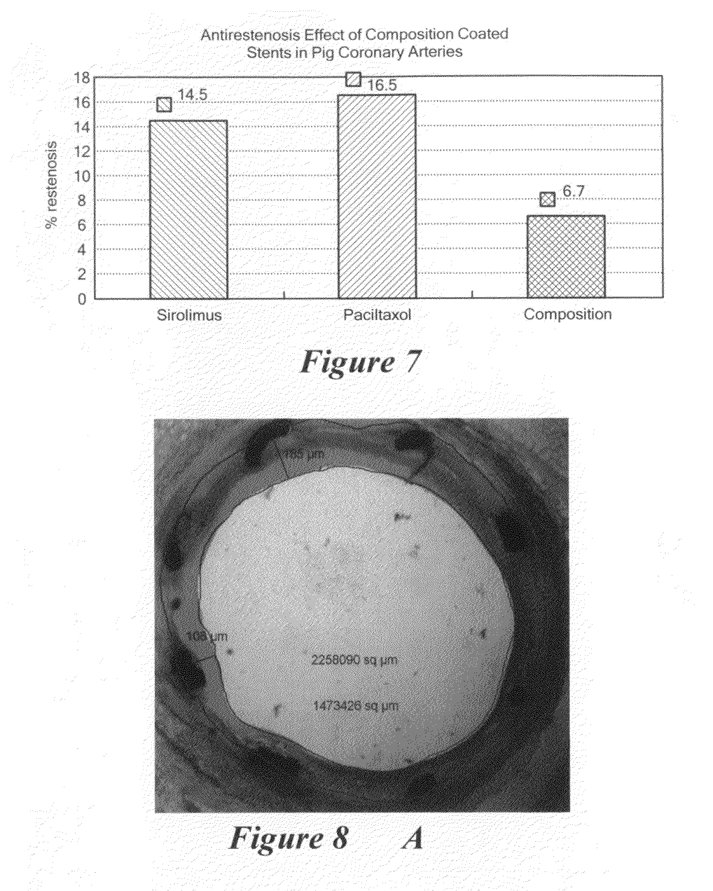

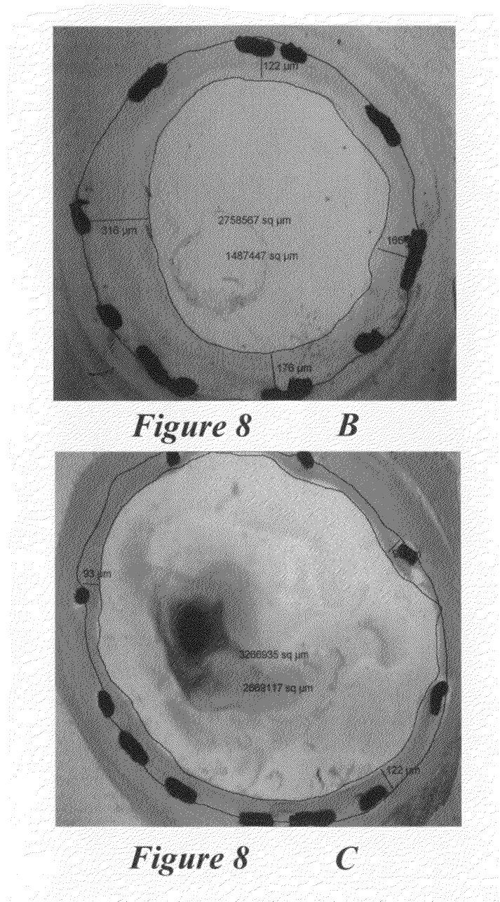

FIG. 7 depicts the differences in the rates of in-stent restenosis among the three experimental groups. The rate of restenosis in sirolimus and paclitaxol coated stents are 14.5% and 15.6% respectively. However, the rate of restenosis in composition coated stents is significantly less (6.7%) than that of stent coated with either sirolimus or paciltaxol alone. FIG. 8 depicts the pathological difference among those three groups. The composition coated stents (C) has the least neointima formation among three groups. The inner wall of stented arteries in the composition coated stent groups was covered by a thin layer of endothelial cells, which is a strong indication of the reendothelialization process taking place. The data from this study demonstrates that composition coated stents promote significantly less restenosis formation than stents coated with either sirolimus or paclitaxol alone at four weeks following porcine coronary implantation.

### EQUIVALENTS AND SCOPE

Those skilled in the art will recognize, or be able to ascertain using no more than routine experimentation, many equivalents to the specific embodiments, described herein. The scope of the present invention is not intended to be limited to the above Description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any cell type; any neuronal cell system; any reporter of synaptic vesicle cycling; any electrical stimulation system; any imaging system; any synaptic vesicle cycling assay; any synaptic vesicle cycle modulator; any working memory modulator; any disorder associated with working memory; any method of use; etc.) can be excluded from any one or more claims, for any reason, whether or not related to the existence of prior art.

### INCORPORATION OF REFERENCES

All publications and patent documents cited in this application are incorporated by reference in their entirety to the same extent as if the contents of each individual publication or patent document were incorporated herein.

What is claimed is:

1. A coating for an implantable or insertable medical device comprising an immunosuppressant agent, an anti-neoplastic agent and one or more polymers, wherein the coating is characterized with sustained-release of the immunosuppressant agent and the anti-neoplastic agent for at least about 4 weeks.

2. The coating of claim 1, wherein said immunosuppressant agent is sirolimus or a prodrug or analog thereof.

3. The coating of claim 2, wherein said sirolimus analog and/or prodrug is selected from the group consisting of zotarolimus, tacrolimus, everolimus, biolimus, pimecrolimus, supralimus, temsirolimus, TAFA 93, invamycin and neuroimmunophilins, and combinations or analogs thereof.

4. The coating of claim 1, wherein said anti-neoplastic agent is paclitaxel or a prodrug or analog thereof.

5. The coating of claim 1, wherein said anti-neoplastic agent is selected from the group consisting of carboplatin, vinorelbine, doxorubicin, gemcitabine, actinomycin-D, cisplatin, camptothecin, 5-fluorouracil, cyclophosphamide, 1-β-D-arabinofuranosylcytosine, and combinations or analogs thereof.

6. The coating of claim 1, wherein the ratio of the immunosuppressant agent and the anti-neoplastic agent, by weight, ranges from 99:1 to 1:99.

7. The coating of claim 6, wherein the ratio of the immunosuppressant agent and the anti-neoplastic agent, by weight, is about 1:1.

8. The coating of claim 1, wherein the immunosuppressant agent and/or the anti-neoplastic agent is present in an amount ranging from about 0.1 μg/mm² to about 5 μg/mm².

9. The coating of claim 1 further comprises an anti-thrombotic agent, an anti-proliferative agent, an anti-inflammatory agent, an anti-migratory agent, an agent affecting extracellular matrix production and organization, an anti-mitotic agent, an anesthetic agent, an anti-coagulant, a vascular cell growth promoter, a vascular cell growth inhibitor, a cholesterol-lowering agent, a vasodilating agent, or an agent that interferes with endogenous vasoactive mechanisms.

10. The coating of claim 1, wherein the one or more polymers comprise a biodegradable polymer.

11. The coating of claim 10, wherein the biodegradable polymer is a polyester polymer.

12. The coating of claim 11, wherein the polyester polymer is selected from the group consisting of poly(D,L-lactide-co-glycolide) (PLGA), polylactides (PLA), Poly(L-lactide) (PLLA), Poly (D,L-lactide) (PDLA), polyglycolides (PGA), and combinations thereof.

13. The coating of claim 11, further comprising a calcium phosphate.

14. The coating of claim 13, wherein the ratio of the polyester polymer and the calcium phosphate ranges from about 99:1 to 1:99.

15. The coating of claim 13, wherein the calcium phosphate is selected from the group consisting of amorphous calcium phosphate (ACP), dicalcium phosphate (DCP), tricalcium phosphate (TCP), pentacalcium hydroxyl Apatite (HAp), tetracalcium phosphate monoxide (TTCP), and combinations thereof.

16. The coating of claim 1, wherein the one or more polymers comprise a nonbiodegradable polymer.

17. The coating of claim 16, wherein the nonbiodegradable polymer is selected from the group consisting of poly-n-butyl methacrylate (PBMA), polyethylene-co-vinyl Acetate (PEVA), poly(styrene-b-isobutylene-b-styrene) (SIBS), and combinations thereof.

18. The coating of claim 1, wherein the immunosuppressant agent and the anti-neoplastic agent are present in the same layer.

19. The coating of claim 1, wherein the immunosuppressant agent and the anti-neoplastic agent are present in different layers.

20. The coating of claim 18, further comprising a cap layer over the layer containing the immunosuppressant agent and the anti-neoplastic agent.

21. The coating of claim 20, wherein the cap layer comprises a biodegradable polymer.

22. An implantable or insertable medical device coated with the coating of claim 1.

23. The implantable or insertable medical device of claim 22 selected from the group consisting of a catheter, a guide wire, a balloon, a filter, a stent, a stent graft, a vascular graft, a vascular patch, or a shunt.

24. The implantable or insertable medical device of claim 23, wherein said device is a stent.

25. The implantable or insertable medical device of claim 24, wherein the stent is a metal stent made from a material selected from the group consisting of stainless steel, nitinol, tantalum, platinum, cobalt alloy, titanium, gold, a biocompatible metal alloy, iridium, silver, tungsten, and combinations thereof.

26. The implantable or insertable medical device of claim 24, wherein the stent is made from a material selected from the group consisting of carbon, carbon fiber, cellulose acetate, cellulose nitrate, silicone, polyethylene teraphthalate, polyurethane, polyamide, polyester, polyorthoester, polyanhydride, polyether sulfone, polycarbonate, polypropylene, polyethylene, polytetrafluoroethylene, polylactic acid, polyglycolic acid, a polyanhydride, polycaprolactone, polyhydroxybutyrate, and combinations thereof.

27. A method of treating a disease or disorder associated with a hyperproliferative condition using the implantable or insertable medical device of claim 22.

28. A method of treating a cardiovascular disease using a stent coated with the coating of claim 1.

29. A drug eluting system comprising:
an implantable or insertable medical device;
a coating comprising an immunosuppressant agent, an anti-neoplastic agent and one or more polymers, wherein the coating is characterized with sustained-release of the immunosuppressant agent and the anti-neoplastic agent for at least about 4 weeks.

30. A method of treating restenosis or other hyperproliferative conditions comprising controlled release of sirolimus and paclitaxel from the surface of an implantable or insertable medical device.

31. The method of claim 30, wherein the restenosis occurs in a blood vessel selected from coronary artery, peripheral artery, brain artery, kidney artery, hepatic artery, bile duct, esophageal artery or bronchial artery.

32. The method of claim 30, wherein the implantable or insertable medical device is selected from the group consisting of a catheter, a guide wire, a balloon, a filter, a stent, a stent graft, a vascular graft, a vascular patch, a shunt, and combinations thereof.
